(19) United States
(12) Patent Application Publication
(10) Pub. No.: US 2018/0313812 A1
(43) Pub. Date: Nov. 1, 2018

(54) METASTATIC CANCER DIAGNOSIS VIA DETECTING pH-DEPENDENT ACTIVATION OF AUTOPHAGY IN INVASIVE CANCER CELLS

(71) Applicants: MOHAMMAD ABDOLAHAD, Tehran (IR); ALIREZA ALIKHANI, Shiraz (IR); Milad Gharooni, Tehran (IR); Hamed Abiri, Tehran (IR); Fatemeh Farokhmanesh, Tehran (IR)

(72) Inventors: MOHAMMAD ABDOLAHAD, Tehran (IR); ALIREZA ALIKHANI, Shiraz (IR); Milad Gharooni, Tehran (IR); Hamed Abiri, Tehran (IR); Fatemeh Farokhmanesh, Tehran (IR)

(73) Assignee: NanoHesgarsazan Salamat Arya, Tehran (IR)

(21) Appl. No.: 16/030,066

(22) Filed: Jul. 9, 2018

Related U.S. Application Data
(60) Provisional application No. 62/530,300, filed on Jul. 10, 2017.

(51) Int. Cl. G01N 33/483; G01N 27/02

(52) U.S. Cl. CPC G01N 33/4836; G01N 27/026

(57) ABSTRACT

A method for detecting a metastasis state of biological cells is disclosed. The method includes seeding a plurality of biological cells onto an array of electrodes of an electrical cell-substrate impedance sensor (ECIS) by dropping a cell suspension including the plurality of biological cells in a cell culture medium onto the array of electrodes, forming a plurality of cultured biological cells attached onto the array of electrodes by maintaining the ECIS in an incubator, reducing pH value of an extracellular media around the plurality of cultured biological cells to a pH value between 6.2 and 6.7 by dropping an acidic solution onto the array of electrodes, activating an intracellular phenomenon due to reducing pH value of the extracellular media around the plurality of cultured biological cells, monitoring an electrical signal of the plurality of cultured biological cells for a pre-determined period of time, and determining metastasis state of the plurality of biological cells based on the monitored electrical signals. The intracellular phenomenon includes one of autophagy phenomenon in metastatic cells, or cell's proliferation reduction and/or apoptosis in non-metastatic cells. Monitoring the electrical signal of the plurality of cultured biological cells includes applying an electrical voltage to the array of electrodes and extracting a set of time-lapse electrical signals from the array of electrodes. Determining metastasis state of the plurality of biological cells includes identifying a metastatic state for the plurality of biological cells by detecting an increasing trend in the set of time-lapse electrical signals over time, where the increasing trend occurs responsive to activation of the autophagy phenomenon. Determining metastasis state of the plurality of biological cells further includes identifying a non-metastatic state for the plurality of biological cells by detecting a decreasing trend in the set of time-lapse electrical signals over time, where the decreasing trend occurs responsive to activation of the cell's proliferation reduction and/or apoptosis phenomenon.

### CROSS-REFERENCE TO RELATED APPLICATION

This application claims the benefit of priority from pending U.S. Provisional Patent Application Ser. No. 62/530,300 filed on Jul. 10, 2017, and entitled “TRACING THE PH DEPENDENT ACTIVATION OF AUTOPHAGY IN CANCER CELLS”, which is incorporated herein by reference in its entirety.

### SPONSORSHIP STATEMENT

This application has been sponsored by Iran Patent Office, which does not have any rights in this application.

### TECHNICAL FIELD

The present disclosure generally relates to cancer diagnosis, and particularly, to distinguishing invasive tumor cells from non-invasive tumor and/or healthy cells by monitoring the electrical impedance change of the cells due to acidity changes of extracellular media.

### BACKGROUND

In contrast to normal cells, cancer cells resist against acidic stress by upregulating autophagy as a survival mechanism to maintain their vital functions. Autophagy activates the acidic stress ion channels (ASIC) in cancer cells. So, a progressive state of the tumor exhibits a direct correlation with its resistance to acidic stresses. Accordingly, evaluating the pH of the cell's microenvironment could be lightening for potentially invasive cancer cells. Different approaches has been applied to measure the pH of cancer involved medium. In vivo measurement of the tumor pH has been carried on by pH-sensitive PET radiotracers, MR spectroscopy and MRI. Such systems are so complicated and expensive that they may not be used before the appearance of clinical signatures of the patient-reducing advanced prognosis. Also, they cannot be used in early diagnosis of cancer which could be achieved by a simple biopsy or pop smear in vitro methods.

Hence, there is a need for a device and method for metastasis diagnosis based on the distinct characteristics of invasive cancer cells at acidic conditions. Additionally, there is a need for a method without any need of functionalization and biomarkers for metastatic cancer diagnosis, where the method is capable of real-time monitoring of the cells behaviors at acidic conditions. Moreover, there is a need for an accurate method for metastasis diagnosis with repeatable processes and results by doing simple procedures.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes an exemplary method for detecting a metastasis state of biological cells. The method may include seeding a plurality of biological cells onto an array of electrodes of an electrical cell-substrate impedance sensor (ECIS) by dropping a cell suspension including the plurality of biological cells in a cell culture medium onto the array of electrodes, forming a plurality of cultured biological cells attached onto the array of electrodes by maintaining the ECIS in an incubator, reducing pH value of an extracellular media around the plurality of cultured biological cells to a pH value between 6.2 and 6.7 by dropping an acidic solution onto the array of electrodes, activating an intracellular phenomenon due to reducing pH value of the extracellular media around the plurality of cultured biological cells, monitoring an electrical signal of the plurality of cultured biological cells for a pre-determined period of time, and determining metastasis state of the plurality of biological cells based on the monitored electrical signals. The intracellular phenomenon may include one of autophagy phenomenon in metastatic cells, or cell's proliferation reduction and/or apoptosis in non-metastatic cells. Monitoring the electrical signal of the plurality of cultured biological cells may include applying an electrical voltage to the array of electrodes and extracting a set of time-lapse electrical signals from the array of electrodes.

In an exemplary implementation, determining metastasis state of the plurality of biological cells based on the monitored electrical signals may include identifying a metastatic state for the plurality of biological cells by detecting an increasing trend in the set of time-lapse electrical signals over time, where the increasing trend may occur responsive to activation of the autophagy phenomenon. In another exemplary implementation, determining metastasis state of the plurality of biological cells based on the monitored electrical signals may include identifying a non-metastatic state for the plurality of biological cells by detecting a decreasing trend in the set of time-lapse electrical signals over time, where the decreasing trend may occur responsive to activation of the cell's proliferation reduction and/or apoptosis phenomenon.

In an exemplary implementation, non-metastatic cells may include at least one of healthy cells, primary cancer cells, and combinations thereof. Furthermore, identifying the non-metastatic state for the plurality of biological cells may include identifying the plurality of biological cells including at least one of healthy cells, primary cancer cells, and combinations thereof.

In an exemplary implementation, monitoring the electrical signal of the plurality of cultured biological cells for the pre-determined period of time may include measuring the set of time-lapse electrical signals from the array of electrodes and recording the set of time-lapse electrical signals measured from the array of electrodes. Measuring the set of time-lapse electrical signals from the array of electrodes may include applying the electrical voltage to the array of electrodes and extracting the set of time-lapse electrical signals from the array of electrodes.

In an exemplary implementation, the set of time-lapse electrical signals may include a set of electrical impedances of the plurality of cultured biological cells. In an exemplary embodiment, the pre-determined period of time may include at least 8 hours after reducing pH value of the extracellular media around the plurality of cultured biological cells. In another exemplary embodiment, the set of time-lapse electrical signals may include a set of electrical impedance values measured every about 2 hours after reducing pH value of the extracellular media around the plurality of cultured biological cells.

In an exemplary implementation, applying the electrical voltage to the array of electrodes may include applying a voltage ranging between 200 mV and 500 mV onto the array of electrodes. In an exemplary embodiment, the electrical voltage may be applied with a frequency ranging between 200 Hz and 100 kHz.

In an exemplary implementation, maintaining the ECIS in the incubator may include maintaining the ECIS with the cell suspension dropped onto the array of electrodes in a CO₂ incubator for a time interval between 2 hours and 5 hours. In an exemplary embodiment, the CO₂ incubator may include about 5% CO₂ and about 95% clean air.

In an exemplary implementation, the array of electrodes may include an array of gold electrodes with a comb-shaped pattern and each electrode of the array of electrodes may include a plurality of silicon nanowires (SiNWs) covered onto each gold electrode. In an exemplary embodiment, the array of electrodes may include a plurality of electrodes with an equal width ranging between 10 μm and 100 μm. In an exemplary embodiment, the array of electrodes may include a first electrode and a second electrode located next to the first electrode. In an exemplary embodiment, a distance between the first electrode and the second electrode may be between about 10 μm and about 100 μm.

In an exemplary implementation, monitoring the electrical signal of the plurality of cultured biological cells for a pre-determined period of time is done through a system, which may include a sensor package including the ECIS, an electrical readout board connected to the ECIS via coaxial wires, and a data processor connected to the electrical readout board via an electrical connector.

In an exemplary embodiment, the electrical readout board may be configured to apply the electrical voltage to the array of electrodes. The electrical readout board may be further configured to extract the set of time-lapse electrical signals from the array of electrodes. The data processor may be configured to record the set of time-lapse electrical signals extracted by the electrical readout board. In an exemplary embodiment, the sensor package may further include a plexiglass cover and the ECIS may be packed in the plexiglass cover.

In an exemplary implementation, a method for metastasis diagnosis may be disclosed. The method may include seeding a plurality of biological cells suspicious to be metastatic onto an array of electrodes of an electrical cell-substrate impedance sensor (ECIS) by dropping a cell suspension including the plurality of biological cells in a cell culture medium onto the array of electrodes, forming a plurality of cultured biological cells attached onto the array of electrodes by maintaining the ECIS in an incubator, reducing pH value of an extracellular media around the plurality of cultured biological cells to a pH value between about 6.2 and about 6.7 by dropping an acidic solution onto the array of electrodes, activating autophagy phenomenon in metastatic cells due to reducing pH value of the extracellular media around the plurality of cultured biological cells, monitoring an electrical signal of the plurality of cultured biological cells for a pre-determined period of time, and diagnosing metastasis by detecting an increasing trend in the set of time-lapse electrical signals over time. Where, the increasing trend may occur responsive to activation of the autophagy phenomenon

In an exemplary implementation, monitoring the electrical signal of the plurality of cultured biological cells for the pre-determined period of time may include applying an electrical voltage to the array of electrodes, and extracting a set of time-lapse electrical signals from the array of electrodes.

In an exemplary implementation, diagnosing metastasis may include detecting an increasing trend in the set of time-lapse electrical signals for a metastatic cell responsive to reducing pH value of the extracellular media around the plurality of cultured biological cells. In an exemplary embodiment, diagnosing metastasis may include detecting a reduction trend over time in the set of time-lapse electrical signals for a non-metastatic cell responsive to activation of a cell's proliferation reduction and/or apoptosis in non-metastatic cells due to reducing pH value of the extracellular media around the plurality of cultured biological cells. In one exemplary embodiment, the non-metastatic cell may include at least one of a normal cell, a primary cancer cell, and combinations thereof.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1 illustrates an exemplary method for metastasis diagnosis, consistent with one or more exemplary embodiments of the present disclosure.

FIG. 2A illustrates a schematic view of an exemplary electrical cell-substrate impedance sensor (ECIS), consistent with one or more exemplary embodiments of the present disclosure.

FIG. 2B illustrates a schematic top view of an exemplary array of electrodes of an exemplary ECIS, consistent with one or more exemplary embodiments of the present disclosure.

FIG. 3 illustrates a schematic implementation of an exemplary system for monitoring an electrical signal from a plurality of cultured biological cells attached onto exemplary array of electrodes of exemplary ECIS, consistent with one or more exemplary embodiments of the present disclosure.

FIG. 4A illustrates a field emission scanning electron microscopy (FESEM) image of exemplary SiNW-covered electrodes array in a comb like array of an exemplary fabricated SiNW-ECIS, consistent with one or more exemplary embodiments of the present disclosure.

FIG. 4B illustrates a FESEM image of a magnified portion of the surface of exemplary fabricated SiNW-ECIS, consistent with one or more exemplary embodiments of the present disclosure.

FIG. 4C illustrates a FESEM image of a more magnified portion of the surface of exemplary fabricated SiNW-ECIS representing a plurality of SiNWs grown and covered on the electrodes, consistent with one or more exemplary embodiments of the present disclosure.

FIG. 5 illustrates a FESEM image of an exemplary MCF7 cell 500 attached to the SiNWs of an exemplary fabricated SiNW-ECIS, consistent with one or more exemplary embodiments of the present disclosure.

FIG. 6 illustrates MTT assay results representing the percentage of cell growth on doped SiNWs and undoped SiNWs relative to a control sample, consistent with one or more exemplary embodiments of the present disclosure.

FIG. 7 illustrates diagrams of the changes in mean electrical impedance of MCF10, MCF7, and MDA-MB468 cells measured at frequency of about 4 kHz after incubation in acidic media with three different pH values of 7.4 (Control), 6.5, and 5.5 at 12 and 24 hours versus 4 hours of culturing time, consistent with one or more exemplary embodiments of the present disclosure.

FIG. 8 illustrates diagrams of the changes in mean electrical capacitance of MCF10, MCF7, and MDA-MB468 cells measured at frequency of about 4 kHz after incubation in acidic media with three different pH values of 7.4 (Control), 6.5, and 5.5 at 12 and 24 hours versus 4 hours of culturing time, consistent with one or more exemplary embodiments of the present disclosure.

FIG. 9A illustrates detection limit profiles of exemplary SiNW-ECIS in sensing the effect of acidic culture media for MCF10 cells, consistent with one or more exemplary embodiments of the present disclosure.

FIG. 9B illustrates detection limit profiles of exemplary SiNW-ECIS in sensing the effect of acidic culture media for MCF7 cells, consistent with one or more exemplary embodiments of the present disclosure.

FIG. 9C illustrates detection limit profiles of exemplary SiNW-ECIS in sensing the effect of acidic culture media for MDA-MB468 cells, consistent with one or more exemplary embodiments of the present disclosure.

FIG. 10A illustrates comparative mean diagrams of Annexin PI results for healthy MCF10 cells in three different pH values of 7.4 (Control), 5.5 and 6.5 after about 24 hours, consistent with one or more exemplary embodiments of the present disclosure.

FIG. 10B illustrates comparative mean diagrams of Annexin PI results for tumorigenic MCF7 cells in three different pH values of 7.4 (Control), 6.5 and 5.5 after about 24 hours, consistent with one or more exemplary embodiments of the present disclosure.

FIG. 10C illustrates comparative mean diagrams of Annexin PI results for metastatic MDA-MB468 cells in three different pH values of 7.4 (Control), 6.5 and 5.5 after about 24 hours, consistent with one or more exemplary embodiments of the present disclosure.

FIG. 11A illustrates optical images of MCF7 cells cultured in three different pH values of 7.4 (left side image), 6.5 (middle image) and 5.5 (right side image), consistent with one or more exemplary embodiments of the present disclosure.

FIG. 11B illustrates optical images of MDA-MB468 cells cultured in three different pH values of 7.4 (left side image), 6.5 (middle image) and 5.5 (right side image), consistent with one or more exemplary embodiments of the present disclosure.

FIG. 12A illustrates Western blotting profile of MCF7 cells incubated in three different pH values of 7.4 (Control), 6.5, and 5.5 based on the expression LC3 associated proteins, consistent with one or more exemplary embodiments of the present disclosure.

FIG. 12B illustrates Western blotting profile of MDA-MB468 cells incubated in three different pH values of 7.4 (Control), 6.5, and 5.5 based on the expression LC3 associated proteins, consistent with one or more exemplary embodiments of the present disclosure.

FIG. 13 illustrates Zymography results based on the expression of MMP2 for MDA-MB468 cells maintained in three different pH values of 7.4 (Control), 6.5, and 5.5 for about 4 hours, consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings. The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

The microenvironmental characteristics of cancer cells may reflect their biological parameters to indicate their phenotype, and pH level is one of such important specifications. For example, a combination of poor vascular perfusion, regional hypoxia, and increased flux of carbons through fermentative glycolysis leads to extracellular acidosis in the medium of cancer cells with extracellular pH values as low as 6.5. Notably, environmental acidification is a good indicator of cancerous transformation as there is overwhelming evidence that corroborate that the most acidic tumors are more likely to be invasive invade and make local metastasis.

The mechanism of such acidification has been discussed elsewhere. Through Pasteur effect, a metabolic switch toward a more glycolytic phenotype, increased reliance on anaerobic metabolism of glucose to lactic acid in cancer cells may occur in cancer cells. Consequently, diffusion limitations and increased production of acid from hypoxic-glycolytic cells, may lead to the increase in proton (H⁺) concentration within the lumen; thereby, causing the interior of the lumen to become highly acidic. Such highly acidic microenvironment may protect cancer cells from immune system control and may enhance the transition probability from an avascular pre-invasive tumor to an invasive malignancy.

Moreover, impedance is frequency dependent electrical resistance which may exhibit a strong correlation with dielectric properties of the materials. Two electrodes may be connected to AC voltage in a known range of frequencies during impedance measurements. Hence, electrical current may pass from the first electrode to the second electrode. Current may interact with a number of cells that may be adhered between the electrodes and a fraction of the current may be blocked by the cells depending on their dielectric properties. A reduced current may indicate increased electrical resistance which may result in an increase of impedance.

Similarly, bio-impedance or biological impedance may be defined as the ability of the biological tissue to impede electric current. Any biological perturbations induced on the cells may affect their dielectric properties and subsequently may change the impedance of a sensor. Hence, the biological effect of the pH changes (as a biological stimulation) may be traced by monitoring the changes in the impedance response of the cells. Cellular impedimetric behavior may be equivalent with an electrical circuit that may include a combination of a capacitor and a resistor.

Herein, an exemplary electrical biosensing method for monitoring the pH dependent behavior of normal and cancer cells by measuring their proliferation dependent electrical resistance (impedance) in acidic media is disclosed. Exemplary method may be used as one way to diagnose invasive tumor cells. The proliferation and mitosis rate may be the main monitoring parameters. Proliferative behavior of healthy and primary cancer cells in acidic media may be compared with invasive cancer cells. Probable activation of autophagy may also be investigated to determine the phenotypic dependent activity of the cells in acidic pH. Disclosed exemplary method that does not require any functionalization and biomarkers, thus, benefits from real-time monitoring of the cell vitality states and repeatable responses after simple washing protocols.

Herein, either “a normal cell” or “a healthy cell” both refer to a healthy cell which is not cancerous, so that “normal cell” and “healthy cell” may be used instead of each other throughout the present disclosure. Additionally, “a metastatic cell”, “a malignant cell”, and “an invasive cell” all refer to a cancerous cell with invasive behavior, so they may be used instead of each other. Moreover, “a primary cancer cell” refers to a non-invasive cancer cell or a non-metastatic cancer cell. In addition, “a non-metastatic cell” refers to either a normal (healthy) cell or a primary cancer cell.

An electrical cell-substrate impedance sensor (ECIS) including an array of silicon nanowires (SiNWs) as electrodes (SiNWs-ECIS) may be fabricated and utilized for one or more steps of exemplary method for early diagnosis of invasive cancer cells. The electrodes may be fabricated of skein SiNWs as an interface for cellular bioelectrical monitoring with 3D interactive surface, which may improve signal extraction from the adhered cells. Great stable physical and chemical properties in weak acids with moderate pH may be other advantages of selecting the SiNWs for 3D bioelectronics approaches in monitoring the cellular acidification.

In an implementation of an exemplary method, cells may be cultured and attached onto the SiNWs of exemplary ECIS for a time period of about 3 hours to about 4 hours in a standard culturing media with normal pH (about 7.4) to cover the array of SiNWs with the cultured cells. Subsequently, the pH of the culturing media may be reduced to a known acidic value (pH of about 6.5) for a known interval of time (about 4 hours). The electrical impedance of cultured cells may be extracted in a time-lapse manner, for example, once an hour for at least about 8 hours after reducing pH. A presence of invasive cancer cells may be diagnosed as a result of monitoring or determining an increasing trend in measured time-laps electrical impedance values over time. Whereas, normal (healthy) cells or primary cancer cells may show a decreasing trend for electrical impedance in response to an acidic condition in extracellular media.

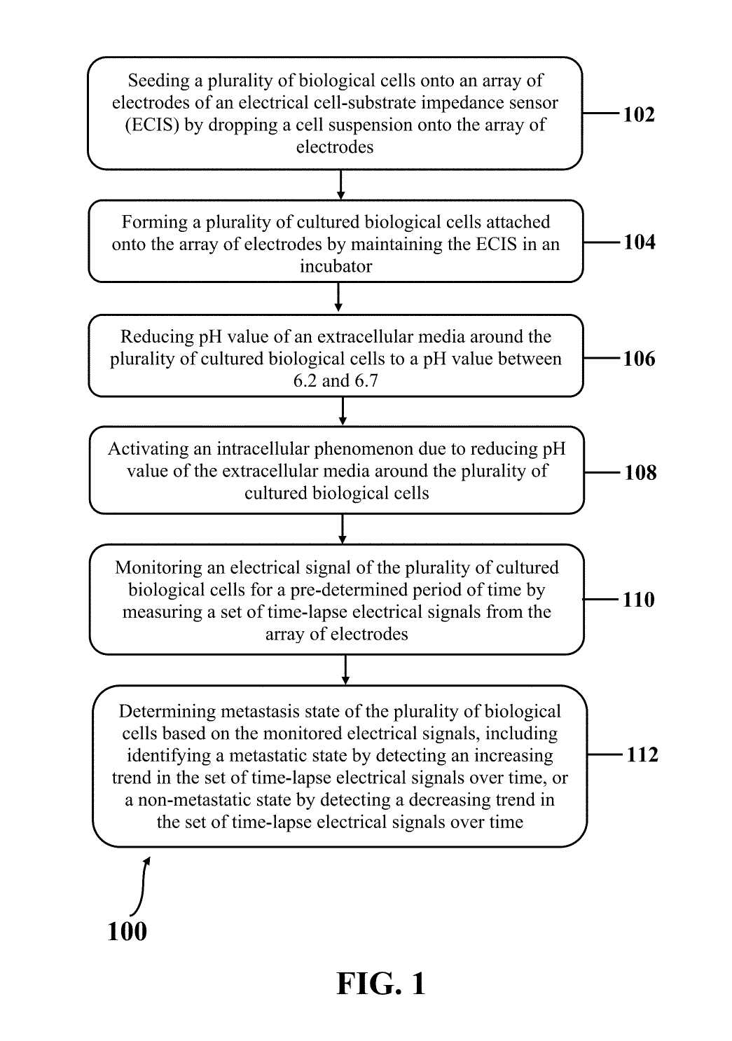

FIG. 1 illustrates an exemplary implementation of method 100 for detecting a metastasis state of biological cells, consistent with one or more exemplary embodiments of the present disclosure. Exemplary method 100 may include seeding a plurality of biological cells onto an array of electrodes of an electrical cell-substrate impedance sensor (ECIS) by dropping a cell suspension onto the array of electrodes (step 102), forming a plurality of cultured biological cells attached onto the array of electrodes by maintaining the ECIS in an incubator (step 104), reducing pH value of an extracellular media around the plurality of cultured biological cells to a pH value between 6.2 and 6.7 by dropping an acidic solution onto the array of electrodes (step 106), activating an intracellular phenomenon due to reducing pH value of the extracellular media around the plurality of cultured biological cells, including activating autophagy phenomenon in metastatic cells, or activating cell's proliferation reduction and/or apoptosis in non-metastatic cells (step 108), and monitoring an electrical signal of the plurality of cultured biological cells for a pre-determined period of time, including applying an electrical voltage to the array of electrodes, and extracting a set of time-lapse electrical signals from the array of electrodes (step 110).

Referring to FIG. 1, exemplary method 100 may further include determining metastasis state of the plurality of biological cells based on the monitored electrical signals (step 112). Determining metastasis state of the plurality of biological cells based on the monitored electrical signals may include one of: identifying a metastatic state for the plurality of biological cells by detecting an increasing trend in the set of time-lapse electrical signals over time, or identifying a non-metastatic state for the plurality of biological cells by detecting a decreasing trend in the set of time-lapse electrical signals over time. The increasing trend may occur responsive to activation of the autophagy phenomenon in metastatic cells due to a reduction of pH in extracellular media, and the decreasing trend may occur responsive to activation of the cell's proliferation reduction and/or apoptosis phenomenon in non-metastatic cells due to a reduction of pH in extracellular media.

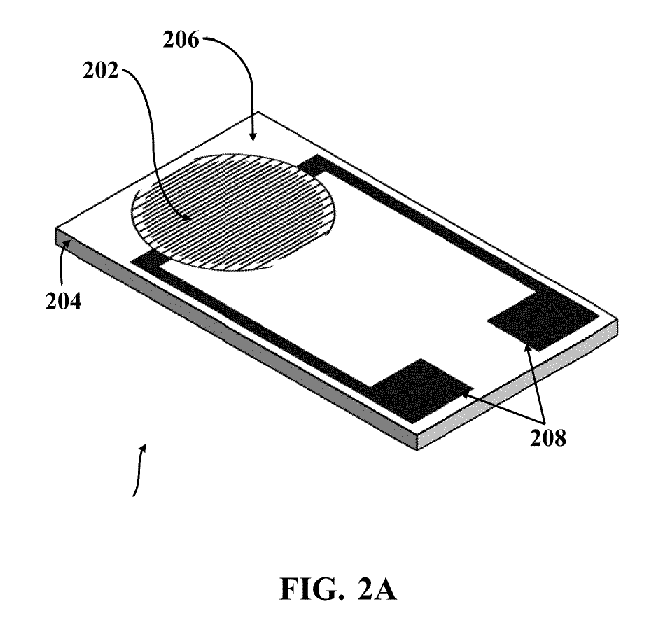

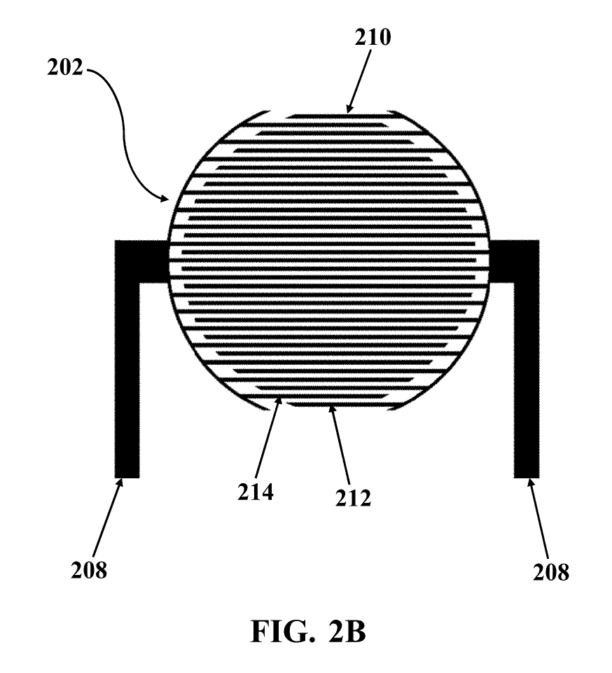

FIG. 2A shows a schematic view of exemplary ECIS 200, consistent with one or more exemplary embodiments of the present disclosure. Exemplary ECIS 200 may include an exemplary array of electrodes 202 patterned and etched onto a surface 206 of a substrate 204. In an exemplary embodiment, substrate 204 may include a silicon wafer or a silicon chip, on which a silicon dioxide layer may be coated. The silicon dioxide layer may be grown on the silicon wafer or a silicon chip.

In an exemplary embodiment, array of electrodes 202 may include an array of gold electrodes with a comb-shaped pattern (an interdigital pattern). Exemplary array of electrodes 202 may be patterned and etched onto surface 206 of exemplary substrate 204 to provide a patterned sensor region in which a plurality of silicon nanowires (SiNWs) are disposed.

In an exemplary embodiment, array of electrodes 202 may include a plurality of SiNWs with enhanced electrical conductivity. The electrical conductivity of the SiNWs may be enhanced via transferring and maintaining exemplary ECIS 200 into a doping furnace. The doping furnace may include a phosphorous doping furnace to enhance the electrical conductivity of nanowires.

Exemplary ECIS 200 may further include electrical connectors 208 which may include gold electrical connectors 208 that may be patterned and etched onto surface 206 of exemplary substrate 204. Exemplary electrical connectors 208 may be configured to transfer an electrical signal to/from exemplary array of electrodes 202.

FIG. 2B shows a schematic top view of exemplary array of electrodes 202 of exemplary ECIS 200, consistent with one or more exemplary embodiments of the present disclosure. In one embodiment, each electrode 210 of exemplary array of electrodes 202 may include a gold electrode covered with a plurality of SiNWs named as “SiNW-covered electrode 210”. So, exemplary array of electrodes 202 may include an array of SiNW-covered electrodes.

It should be noted that the plurality of SiNWs may act as a plurality of bioelectrodes that may configured to attach to a biological cell in order to apply an electrical stimulation to the biological cell, or extract an electrical signal (response) from the attached biological cell. The gold electrode may be a catalyst layer for growing the plurality of SiNWs thereon. In an exemplary embodiment, each SiNW-covered electrode 210 of exemplary array of electrodes 202 may include a tooth of exemplary comb-shaped array of electrodes 202.

Referring to FIG. 2B, exemplary array of electrodes 202 may include a plurality of electrodes with an equal width ranging between about 10 μm and about 100 μm. In an exemplary embodiment, exemplary array of electrodes 202 may include a first electrode 212 and a second electrode 214 located next to the first electrode 212. A distance between the first electrode 212 and the second electrode 214 may be in a range between about 10 μm and about 100 μm.

Referring back to FIG. 1, Step 102 may include seeding the plurality of biological cells onto exemplary array of electrodes 202 of exemplary ECIS 200 by dropping the cell suspension onto exemplary array of electrodes 202. The cell suspension may include the plurality of biological cells in a cell culture medium.

In an exemplary embodiment, the cell suspension may include one of a plurality of healthy cells, a plurality of primary cancer cells, and a plurality of metastatic cancer cells. In an exemplary embodiment, the cell suspension may include a cell line that may include one of a healthy cell line, a primary cancer cell line, and a metastatic cancer cell line. In an exemplary embodiment, the cell suspension may further include a cell culture medium, for example, a Roswell Park Memorial Institute-1640 (RPMI-1640) medium or Dulbecco's Modified Eagle's medium (DMEM).

Step 104 may include forming the plurality of cultured biological cells attached onto exemplary array of electrodes 202 by maintaining exemplary ECIS 200 in the incubator. The plurality of biological cells may be cultured on exemplary array of electrodes 202.

In an exemplary implementation, maintaining exemplary ECIS 200 in the incubator may include maintaining exemplary ECIS 200 with the cell suspension dropped onto the array of electrodes in a CO₂ incubator. Exemplary ECIS 200 may be maintained in the CO₂ incubator for a time interval between about 2 hours and about 5 hours. The CO₂ incubator may include about 5% CO₂ and about 95% clean air.

In an exemplary implementation, the plurality of biological cells may be cultured on the plurality of SiNWs; thereby, resulting in forming the plurality of cultured biological cells attached on the plurality of SiNWs. The attachment between the plurality of cultured biological cells and the plurality of SiNWs may result in an ability of exemplary ECIS 200 for accurately transferring or measuring electrical signals to/from the plurality of cultured biological cells via the plurality of SiNWs acting as a plurality of nanostructured electrodes covered on exemplary array of electrodes 202.

Step 106 may include reducing pH value of the extracellular media around the plurality of cultured biological cells to a pH value between about 6.2 and about 6.7 by dropping an acidic solution onto exemplary array of electrodes 202 by adding acidic solution may be added onto exemplary array of electrodes 202.

In an exemplary implementation, the acidic solution may include a diluted solution of HCl. For example, the acidic solution may include a diluted solution of HCl with a concentration of less than about 2 μM, for example, about 1.25 μM.

Step 108 may include activating the intracellular phenomenon due to reducing pH value of the extracellular media around the plurality of cultured biological cells. The intracellular phenomenon in metastatic cells may include autophagy phenomenon occurring for metastatic cells. The intracellular phenomenon may include cell's proliferation reduction and/or apoptosis in non-metastatic cells.

In an exemplary embodiment, reducing pH value of the extracellular media around the plurality of cultured biological cells attached to exemplary array of electrodes 202 to a pH value between about 6.2 and about 6.7 (step 106) may result in different phenomena via completely different pathways in metastatic cells in comparison with non-metastatic cells. If the plurality of cultured biological cells include a plurality of metastatic cells, an autophagy phenomenon may be activated, so that they may survive at moderate low pH values of between about 6.2 and about 6.7. On the other hand, if the plurality of cultured biological cells include a plurality of non-metastatic cells, an apoptosis or a reduction cell's proliferation may be activated. Autophagy may be activated as a protector pathway for metastatic cells at moderate low pH values of between about 6.2 and about 6.7. Activating autophagy in metastatic cells may cause an increase in an electrical impedance measured from the plurality of cultured biological cells, which may be measured and monitored using exemplary array of electrodes 202.

In an exemplary embodiment, non-metastatic cells may include at least one of healthy cells, primary cancer cells, and combinations thereof. Reducing pH value of the extracellular media around the plurality of cultured biological cells attached to exemplary array of electrodes 202 to a pH value between about 6.2 and about 6.7 (step 106) may result in a reduction in cell's proliferation for primary cancer cells. Moreover, reducing pH value of the extracellular media around the plurality of cultured biological cells attached to exemplary array of electrodes 202 to a pH value between about 6.2 and about 6.7 (step 106) may result in a cell apoptosis for healthy cells. Both cell's proliferation reduction and/or apoptosis in non-metastatic cells (healthy cells and/or primary cancer cells) may cause a reduction in an electrical impedance measured from the plurality of cultured biological cells, which may be measured and monitored using exemplary array of electrodes 202.

Step 110 may include monitoring electrical signals of the plurality of cultured biological cells for a pre-determined period of time that may be done by measuring a set of time-lapse electrical signals. Monitoring the electrical signal of the plurality of cultured biological cells for the pre-determined period of time may include applying an electrical voltage to the array of electrodes and extracting the set of time-lapse electrical signals from the array of electrodes.

In an exemplary implementation, monitoring the electrical signal of the plurality of cultured biological cells for the pre-determined period of time may include measuring the set of time-lapse electrical signals from the array of electrodes and recording the set of time-lapse electrical signals measured from the array of electrodes. Measuring the set of time-lapse electrical signals from the array of electrodes may include applying the electrical voltage to the array of electrodes and extracting the set of time-lapse electrical signals from the array of electrodes.

In an exemplary implementation, monitoring the electrical signal of the plurality of cultured biological cells for the pre-determined period of time may further include tracing the set of time-lapse electrical signals to determine a trend of variations of the set of time-lapse electrical signals.

In an exemplary implementation, the set of time-lapse electrical signals may include a set of electrical impedances of the plurality of cultured biological cells. In an exemplary implementation, the pre-determined period of time may include at least about 8 hours after reducing pH value of the extracellular media around the plurality of cultured biological cells. In an exemplary embodiment, the set of time-lapse electrical signals may include a set of electrical impedance values measured at every about 2 hours after reducing pH value of the extracellular media around the plurality of cultured biological cells.

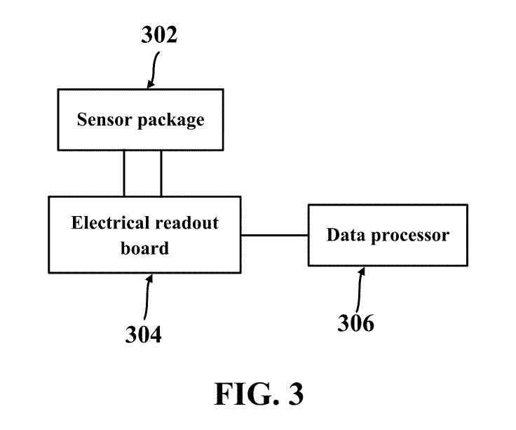

In an exemplary implementation, monitoring the electrical signal of the plurality of cultured biological cells may be done through a system. FIG. 3 shows a schematic implementation of an exemplary system 300 for monitoring the electrical signals of the plurality of cultured biological cells attached onto exemplary array of electrodes 202 of exemplary ECIS 200, consistent with one or more exemplary embodiments of the present disclosure.

Referring to FIG. 3, exemplary system 300 may include a sensor package 302, an electrical readout board 304, and a data processor 306. Exemplary sensor package 302 may include exemplary ECIS 200 and a plexiglass cover (not illustrated). Exemplary ECIS 200 may be placed and packed within the plexiglass cover. Exemplary ECIS 200 may be sealed with a biograde silicon rubber tube within the plexiglass cover. Exemplary ECIS 200 within exemplary sensor package 302 may be connected to electrical readout board 304 via coaxial wires attached to exemplary electrical connectors 208 of ECIS 200 (FIGS. 2A and 2B).

In an exemplary implementation, electrical readout board 304 may be configured to apply the electrical voltage to exemplary array of electrodes 202. In an exemplary embodiment, electrical readout board 304 may further configured to extract the set of time-lapse electrical signals from exemplary array of electrodes 202.

In an exemplary implementation, applying the electrical voltage to exemplary array of electrodes 202 may include applying a voltage ranging between about 200 mV and about 500 mV onto exemplary array of electrodes 202. The electrical voltage may be applied onto exemplary array of electrodes 202 with a frequency ranging between about 200 Hz and about 100 kHz.

In an exemplary implementation, data processor 306 may be connected to electrical readout board 304 via an electrical connector, for example, an electrical wire. Exemplary data processor 306 may be configured to record the set of time-lapse electrical signals extracted by electrical readout board 304 from exemplary array of electrodes 202.

Step 112 may include determining metastasis state of the plurality of biological cells based on the monitored electrical signals. In detail, step 112 may include identifying a metastatic state for the plurality of biological cells by detecting an increasing trend in the set of time-lapse electrical signals over time or identifying a non-metastatic state for the plurality of biological cells by detecting a decreasing trend in the set of time-lapse electrical signals over time.

In an exemplary implementation, the increasing trend may occur responsive to activation of the autophagy phenomenon in metastatic cells in step 108 due to reduction of pH in extracellular media in step 106. So, detecting an increasing trend for the set of time-lapse electrical signals over time after reducing pH in extracellular media may be an indicator or criterion for a dominant presence of metastatic cells among the plurality of biological cells.

In an exemplary implementation, the decreasing trend may occur responsive to activation of the cell's proliferation reduction and/or apoptosis phenomenon in non-metastatic cells in step 108 due to a reduction of pH in extracellular media in step 106. If the plurality of biological cells include non-metastatic cells, a decreasing trend may be detected for the set of time-lapse electrical signals over time after reducing pH in extracellular media. In an exemplary embodiment, identifying the non-metastatic state for the plurality of biological cells may include identifying the plurality of biological cells comprising at least one of healthy cells, primary cancer cells, and combinations thereof.

In some implementations, method 100 may be utilized for metastasis diagnosis. Exemplary method 100 may include seeding a plurality of biological cells suspicious to be metastatic onto an array of electrodes of an electrical cell-substrate impedance sensor (ECIS) by dropping a cell suspension including the plurality of biological cells in a cell culture medium onto the array of electrodes, forming a plurality of cultured biological cells attached onto the array of electrodes by maintaining the ECIS in an incubator, reducing pH value of an extracellular media around the plurality of cultured biological cells to a pH value between about 6.2 and about 6.7 by dropping an acidic solution onto the array of electrodes, activating autophagy phenomenon in metastatic cells due to reducing pH value of the extracellular media around the plurality of cultured biological cells, monitoring an electrical signal of the plurality of cultured biological cells for a pre-determined period of time, and diagnosing metastasis by detecting an increasing trend in the set of time-lapse electrical signals over time. Where, the increasing trend may occur responsive to activation of the autophagy phenomenon

In an exemplary implementation, monitoring the electrical signal of the plurality of cultured biological cells for the pre-determined period of time may include applying an electrical voltage to the array of electrodes, and extracting a set of time-lapse electrical signals from the array of electrodes.

In an exemplary implementation, diagnosing metastasis may include detecting an increasing trend in the set of time-lapse electrical signals for a metastatic cell responsive to reducing pH value of the extracellular media around the plurality of cultured biological cells. In an exemplary embodiment, diagnosing metastasis may include detecting a reduction trend over time in the set of time-lapse electrical signals for a non-metastatic cell responsive to activation of a cell's proliferation reduction and/or apoptosis in non-metastatic cells due to reducing pH value of the extracellular media around the plurality of cultured biological cells. In one exemplary embodiment, the non-metastatic cell may include at least one of a normal cell, a primary cancer cell, and combinations thereof.

### Example 1: Fabrication of SiNWs Impedance Sensor (SiNW-ECIS)

In this example, exemplary ECISs with SiNWs electrodes array (SiNW-ECIS) similar to exemplary ECIS 200 were fabricated. Silicon wafers, which were used as substrates, were cleaned through standard RCA#1 cleaning method (NH₄OH:H₂O₂:H₂O solution and volume ratio of 1:1:5, respectively). Subsequently, a thin layer of SiO₂ with a thickness of about 300 nm was grown on the substrate by wet oxidation furnace. A thin layer of gold (Au) with a thickness of about 10 nm was coated on SiO₂ layer by a sputtering system at a pressure of about 20 mTorr as a catalyst layer. The gold layer was patterned and the design of the sensor transferred to the substrate. Au-covered samples were located in a low pressure chemical vapor deposition (LPCVD) system with a quartz tube chamber. The growth of silicon nanowires (SiNWs) was a two-step process named as graining and growth. During the graining, a thermal annealing at 450° C.-550° C. for about 30 minutes at the presence of argon (Ar) was carried out which resulted in the catalyst graining and formation of gold nano-sized islands. During the growth step, a mixture of high purity silane (SiH₄) as Si source and Ar as a carrier and dilution gases were introduced to the chamber. Silicon crystalline nanostructures were formed on top of the catalyst islands in the patterned region followed by breaking of the silane to Si and Si—H free radicals.

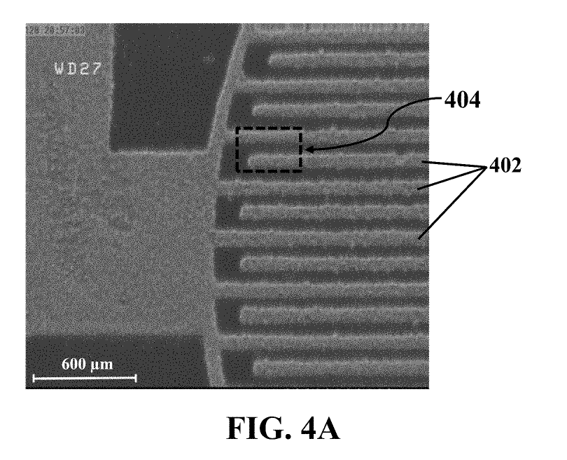

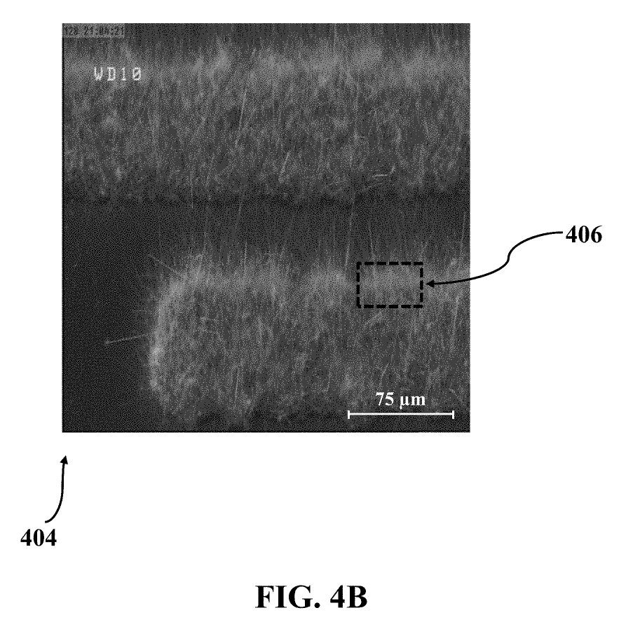

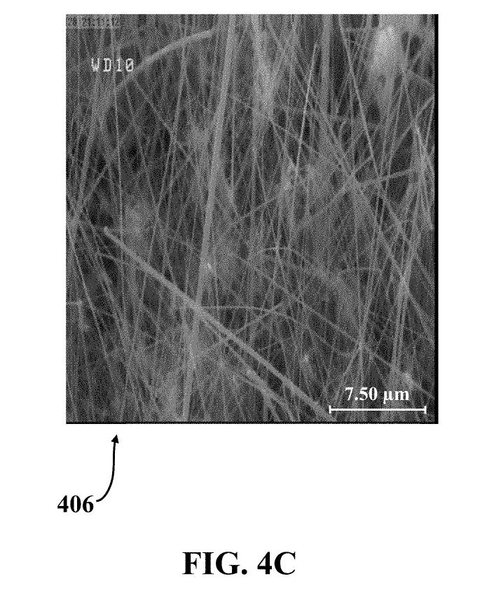

FIG. 4A shows a field emission scanning electron microscopy (FESEM) image of exemplary SiNW-covered electrodes array 402 in a comb like array of an exemplary fabricated SiNW-ECIS similar to exemplary ECIS 200, consistent with one or more exemplary embodiments of the present disclosure. FIG. 4B shows a FESEM image of a magnified portion 404 of the surface of exemplary fabricated SiNW-ECIS, consistent with one or more exemplary embodiments of the present disclosure. Referring to FIGS. 4A and 4B, the width of each electrode of SiNW-covered electrodes array 402 may be about 60 μm. Moreover, a distance between each two adjacent electrodes of SiNW-covered electrodes array 402 may be about 60 μm. These ranges of size may be in a desired range for ECIS applications. FIG. 4C shows a FESEM image of a more magnified portion 406 of the surface of exemplary fabricated SiNW-ECIS representing a plurality of SiNWs grown and covered on the electrodes, consistent with one or more exemplary embodiments of the present disclosure.

### Example 2: Cell Culture and Seeding

In this example, normal cell line (MCF10 non-cancerous breast epithelial cell line), primary cancerous cell line (MCF7 human breast cancer cell line), and metastatic cell line (MDA-MB-468 human breast cancer cell line) were obtained from a standard cell bank, which were isolated from normal sites, grades I and IV of human breast tumors, respectively. The cell lines were held at about 37° C. in an incubator (about 5% CO₂, about 95% air) in RPMI-1640 medium supplemented with about 5% fetal bovine serum, and about 1% penicillin/streptomycin for MCF7 and MDA-MB468 cells and in DMEM-F12 supplemented with about 10% horse serum, about 1% antibiotic/antimitotic solution, about 0.2% NaHCO₃, insulin (about 5 μg/ml), EGF (about 10 ng/ml) and hydrocortisone (about 1 μg/ml) for MCF10 cells. The fresh medium was replaced every other day. Standard cell culture methods were used for cell propagation. The cells were counted and suspended in about 100 μl of respective culture media for monoculture experiments before being introduced into a cloning cylinder containing exemplary SiNW-ECIS, which was fabricated in accordance with Example 1. The seeded cells covered about 50% of the surface of exemplary SiNW-ECIS to let cells have a plenty of space for mitosis. But the initial concentration of dropped normal cells was further because the apoptosis and growth suppression are more probable for normal cells in respect to cancer cells in similar culturing parameters. So the impedance of normal cells-covered exemplary SiNW-ECIS would start from higher values (because of further amount of pre-dropped cells). The cell density was set at about 2000 cells/100 μl for the monoculture experiments.

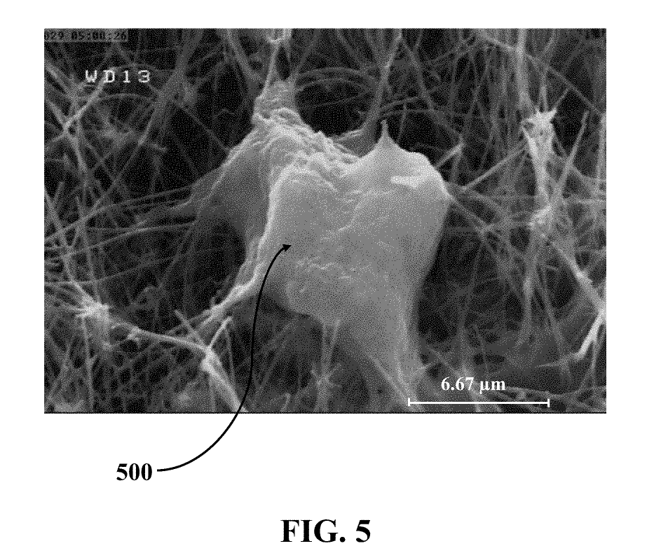

FIG. 5 shows a FESEM image of an exemplary MCF7 cell 500 attached to the SiNWs of an exemplary fabricated SiNW-ECIS, consistent with one or more exemplary embodiments of the present disclosure. A good attachment between MCF7 cell 500 and SiNWs may be observed due to an improved surface for cells culture provided by SiNWs. A three-dimensional (3D) interactive surface between the cultured MCF7 cell 500 and the SiNW electrodes could be observed.

MTT Assay:

An important positive impact of exemplary SiNWs electrodes may be their biocompatibility, which was investigated by 3-(4,5-dimethylthiazol-2-yl)-2,5-diphenyltetrazolium bromide (MTT) assay. The viability of the cells seeded on SiNWs surface was experimented by MTT assay for both doped and undoped SiNWs. After about 24 hours of incubation, about 20 ml of MTT (about 5 mg/ml) was added to each well. The cells were incubated at about 37° C. for about 3 hours. Thereafter, the medium was taken away and the insoluble formazan crystals were dissolved in about 200 ml of dimethylsulfoxide (DMSO). The absorbance was measured at about 490 nm by Microplate Reader. The results were expressed as the percentage of cell growth relative to a control sample.

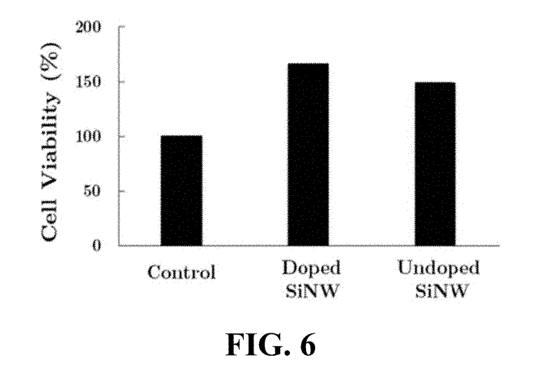

FIG. 6 shows MTT assay results representing the percentage of cell growth on doped SiNWs and undoped SiNWs relative to the control sample, consistent with one or more exemplary embodiments of the present disclosure. The biocompatibility of the SiNWs may be observed according to the high cell viability on both doped and undoped SiNWs.

### Example 3: PH Dependent Electrical Impedance of the Cells Measured by SiNW-ECIS

In this example, the electrical impedances of normal and malignant cells attached onto the SiNWs of exemplary fabricated SiNW-ECIS were measured by an exemplary impedance meter board similar to electrical readout board 304 that was designed and prepared in-house for impedance measurement purposes. Exemplary Impedance meter board was connected through coaxial wires to exemplary SiNW-ECIS including the attached normal and malignant cells onto the SiNWs. Measurements were performed with an applied voltage of about 200 mV. The real time measurements of cellular bioelectrical functions were recorded at desired frequencies (about 0.4 kHz-400 kHz) in the known interval of times. DMEM (including Na⁺, K⁺, Mg²⁺, Ca²⁺ and etc. ions and amino acid, glucose and etc. buffers) was used as cells' carrier solution. The pH was controlled by low concentrations of HCl with the assistance of a pH meter. The electrical responses (electrical impedance (kΩ) and capacitance (associated with phase, Φ) of the cells) were extracted and monitored by 10 times measurements at each frequency and at three levels of pH of about 7.4 (Control), 6.5, and 5.5. To eliminate the effect of medium, the media solution was refreshed before each measurement.

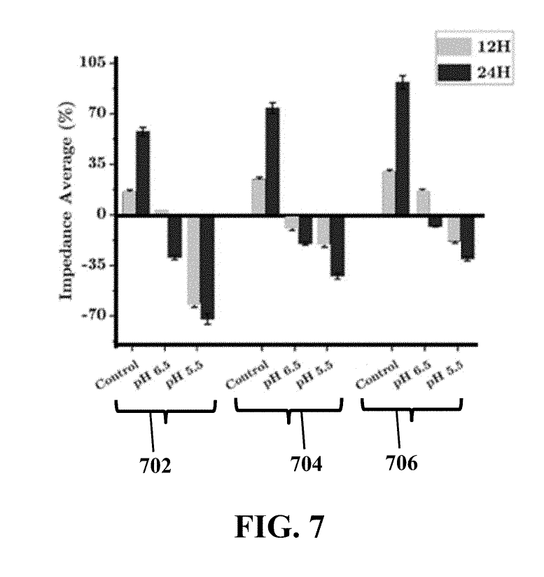

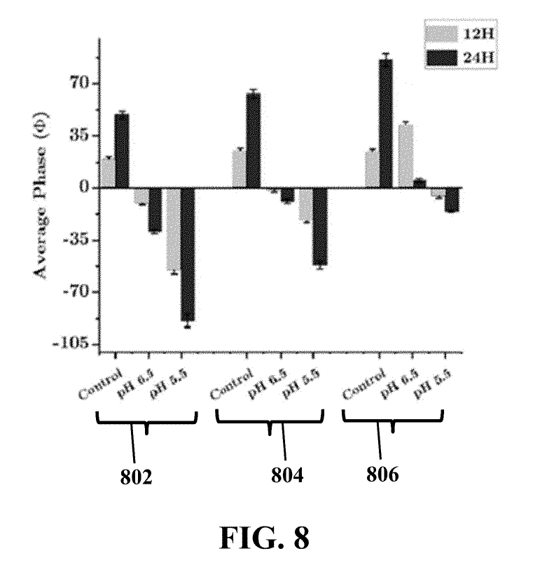

FIG. 7 shows diagrams of the changes in mean electrical impedance of MCF10 cells (columns designated by 702), MCF7 cells (columns designated by 704), and MDA-MB468 cells (columns designated by 706) measured at frequency of about 4 kHz after incubation in acidic media with three different pH values of 7.4 (Control), 6.5, and 5.5 at 12^{th} and 24^{th} hours versus 4^{th} hours of culturing time, consistent with one or more exemplary embodiments of the present disclosure. FIG. 8 shows diagrams of the changes in mean electrical capacitance of MCF10 cells (columns designated by 802), MCF7 cells (columns designated by 804), and MDA-MB468 cells (columns designated by 806) measured at frequency of about 4 kHz after incubation in acidic media with three different pH values of 7.4 (Control), 6.5, and 5.5 at 12 and 24 hours versus 4 hours of culturing time, consistent with one or more exemplary embodiments of the present disclosure.

Referring to FIG. 7, the changes in mean electrical impedance (resistance) of MCF7 cells cultured on SiNWs electrodes in normal and acidic pH values (6.5, 5.5) at 12^{th} and 24^{th} hours versus 4^{th} hours of culturing time may be observed. Resistance increment in Control cells corroborated their progressed proliferative behavior meanwhile reduction in the resistance of the acidified cells exhibited a strong correlation with pH dependent acidosis and loses in the vital behavior of MCF7 cells. The electrical resistance of the cells' layer incubated in normal pH (7.4) at 4^{th} hours after the start of culturing may be assumed as normal reference resistance. In normal pH, at the first 8 hours, the cells showed a minor increase in their resistance because of the proliferation of cancer cells. In next intervals of time, the resistance continued the increasing regime due to the growth and mitosis of the cells which increased the dielectric media covered on the electrodes. In pH=about 6.5, the MCF7 cells were affected by the lower pH of the medium. So they exhibited about 30% to about 50% reduction in electrical resistance at 12^{th} and 24^{th} hours. Such reduction reached to about 70% and about 90% when the cells were incubated at pH=about 5.5. This means that the acidic pH activated the apoptotic pathways on MCF7 cells.

Referring again to FIG. 7, the impedance of MDA-MB468 cells was also increased in Control pH (7.4), but acidic media with pH of about 6.5 couldn't reduce the impedance of MDA-MB468 cells in first 12 hours, which corroborated the activation of autophagy in such cells. The reduction of the impedance of the cells started from 12^{th} hours was a corroboration for activation of apoptotic pathways. Also, severe reduction of the mean impedance in MDA-MB468 cells incubated in pH of about 5.5 could be related to the activation of self-eating in such cells in acidic media.

Similarly, the capacitive behavior of cells in the mentioned pH values was determined and presented in FIG. 8. A different trend for capacitance changes in MDA-MB468 cells maintained in pH 6.5 were recorded with respect to two other types of the cells (MCF7 and MCF10 cells). As a result, just the resistance and capacitance of MDA-MB468 cells exhibited an increment in moderate acidic media (pH of about 6.5) after about 12 hours. The mechanism of autophagy may be activated in maintaining the viability of metastatic cells during being exposed to acidic stress. In acidic media with moderate acidic pH (about 6.5), Autolysosome produced by Autophagosome may be connected to the cells and meanwhile, some metastatic function might be lost, the metastatic cells may be survived in moderate acidic media. In lower pH of about 5.5, the entrance of Autolysosome into the cells may result in cell self-eating and death induced by the autophagy activation. So the function of autophagy in surviving or killing the cell in acidic media is pH dependent.

Detection Limit:

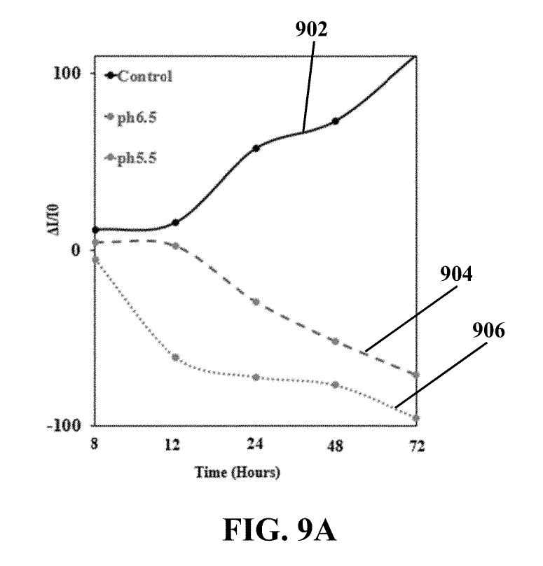

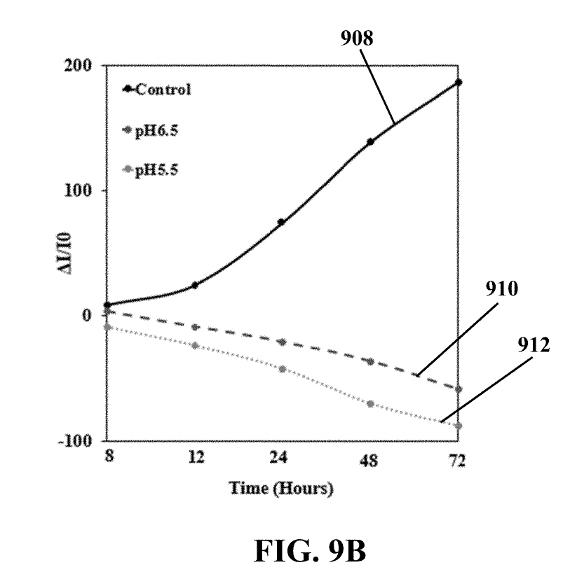

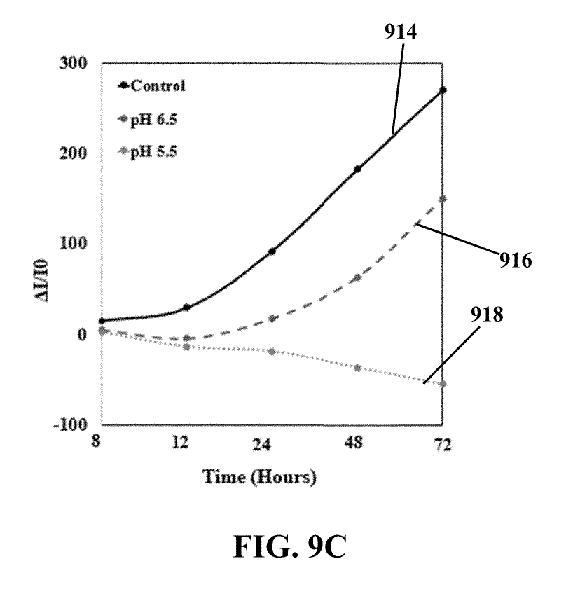

FIGS. 9A-9C show detection limit profiles of exemplary SiNW-ECIS in sensing the effect of acidic culture media for all three types of assayed breast cells, including MCF10 (FIG. 9A), MCF7 (FIG. 9B), and MDA-MB468 (FIG. 9C), consistent with one or more exemplary embodiments of the present disclosure. The detecting resolution followed the formulated regime is presented in each panel of FIGS. 9A-9C. After 8^{th} hour, all of Control cells (shown by curves 902, 908, and 914) continued their growth and proliferation observed in the increased current blocking from primary value (ΔI/I0). Curves 904, 910, and 916 represent detection limit profiles in moderate acidic media with pH of about 6.5, and curves 906, 912, and 918 represent detection limit profiles in high acidic media with pH of about 5.5.

It may be observed from FIGS. 9A-9C that at least about 4 hours are required to observe the electrical response of exemplary SiNW-ECIS after attachment of the cells to the SiNW-covered electrodes. Moreover, the pH dependent distinguished response of deicing was recordable at 12^{th} hour (about 8 hours after attachment) in which the slope of the sensitivity profile became changed in acidic pH for all three type of the cells.

FIGS. 9A-9C reveal that the differential changes in blocking current by cells (ΔI/I0) may be related to time. Due to differences in cells dialectical features, (ΔI/I0) is directly related to Δ_{impedance} and in every time added normalized impedance in percent. The slope of the equation in every sample revealed the ratio of current blocking and in the simple present ratio of cell's growth. MDA-MB468 profile (curve 916) in the low acidic media (pH=about 6.5) has a positive slope which reveals that the impedance in this condition increases with a slope lower than Control sample (curve 914). Time evolution after spreading stages of the cultured cells, resulted in progressed cellular proliferation which would increase the current blocking ability due to increased filling factor of the surface by enhanced cellular layer. This would enhance the absolute value of ΔI/I0 as presented. Additionally, about 2×10⁴ cells were tested in each assay but the dynamic range of the sensor could be increased to about 10⁵ cells in which the surface of the device got saturated. Hence, due to the mitotic rate of cancer cells, preconcentration of about 2×10⁴ and monitoring the responses for about 72 hours could result in non-saturated and desired achievements on the effect of the pH.

### Example 4: ANVPI Analysis to Investigate pH Dependent Behavior in Normal and Cancer Cells

To investigate the effect of reduced pH in probable acidosis of primary cancer cells, individual samples of MCF10A, MCF7 and MDAMB468 cells were maintained in pH 5.5 and 6.5 for about 4 hours, and subsequently incubated in normal pH (7.4) for about 24 hours. ANPI assays then were experimented to evaluate the pH induced cells apoptosis by comparing with the Control cells. Accordingly, percentages of apoptotic and necrotic cells were assessed via FACS analysis of Annexin V-FITC and Propidium Iodide (PI)-stained cells. Measurements were carried out using an apoptosis detection kit. In brief, the cells were washed with PBS and suspended in about 500 μL total volume with about 490 μL binding buffer, about 5 μL PI and about 5 μL Annexin V-FITC. After about 15 minutes incubation in the dark at room temperature, cells were tested for Annexin V binding within about 2 hours using flow cytometry. Annexin PI was used as a biological assay to detect the percent of the assayed cells in Live, Early apoptotic, late apoptotic, and necrotic stages.

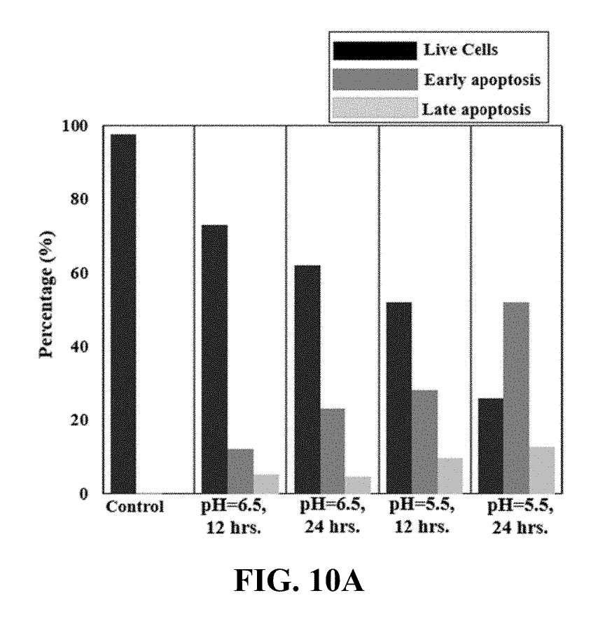

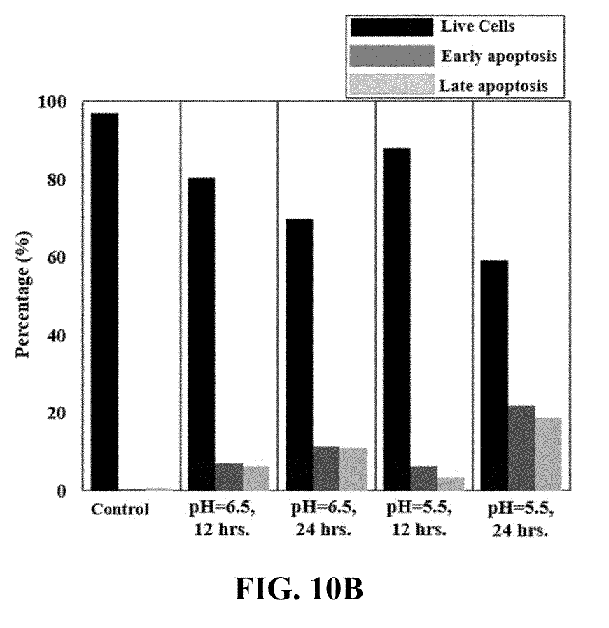

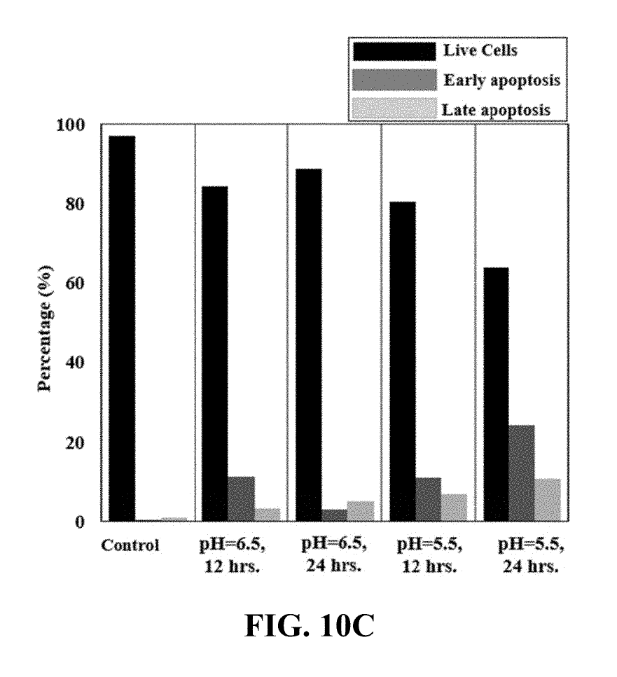

FIGS. 10A-10C show comparative mean diagrams of Annexin PI results for healthy MCF10 cells (FIG. 10A), tumorigenic MCF7 cells (FIG. 10B), and metastatic MDA-MB468 cells (FIG. 10C) in three different pH values of 7.4 (Control), 6.5 and 5.5 after about 24 hours, consistent with one or more exemplary embodiments of the present disclosure. The ANVPI result showed that MCF7 cell line entered to the late apoptosis due to acidic pH (about 22.5% in early and late apoptosis in pH 6.5 and about 40% in early and late apoptosis in pH 5.5 after 24 hours). Whereas, MDA-MB468 resisted against apoptosis in pH 6.5 (about 10% in early and late apoptosis in pH 6.5 and about 35% in early and late apoptosis in the pH 5.5 after 24 hours).

ANPI results revealed that the increased population of the MCF10 cells entered to both early and late apoptosis by about 24 hours maintaining in pH=6.5. Also, the fraction of apoptotic cells was further in pH 5.5. It is worth noting that the ratio of apoptotic cells, observed at 12^{th} hour, increased about 30% at 24^{th} hour for the cells incubated at pH 6.5 and increased about 50% at 24^{th} hour for the cells incubated at pH 5.5.

The ANPI results of MCF7 cells revealed that the meaningful decrease in the fraction of live cells after maintaining in pH=6.5. Also, the cells had been in early apoptotic entered to late apoptotic and subsequently, the late apoptotic cells entered to necrotic state from 12^{th} hour to 24^{th} hour. In pH 5.5, MCF7 cells severely were affected by acidosis and significant increase in the fraction of apoptotic cells are noticeable after about 12 hours.

The comparative ANPI results taken from MDA-MB cells presented a minor decrease in the population of live cells 12 hours and 24 hours after being acidified in the culture media with pH=6.5. The measured decreased fraction in live cells from 12^{th} hour to 24^{th} hour was equal to the minor increment in early apoptotic cells. Also, the reduced fraction of late apoptotic cells revealed no significant entrance of early apoptotic cells to the late apoptotic stage. These results indicated that the MDAMB cells had been cultured in pH=6.5, maintained their vitality by activating the autophagy. Autophagy might be the main function that plays the key role in different behavior of MDA-MB468 invasive cancer cells from other cells in pH=6.5. Increase in apoptotic index of the MDA-MB468 cells had been incubated in pH=5.5, indicated that the cells couldn't protect themselves from acidosis in lower pH values and some secondary phenomena such as autophagy based self-eating would be activated in these acidic conditions.

Optical microscopy image analysis:

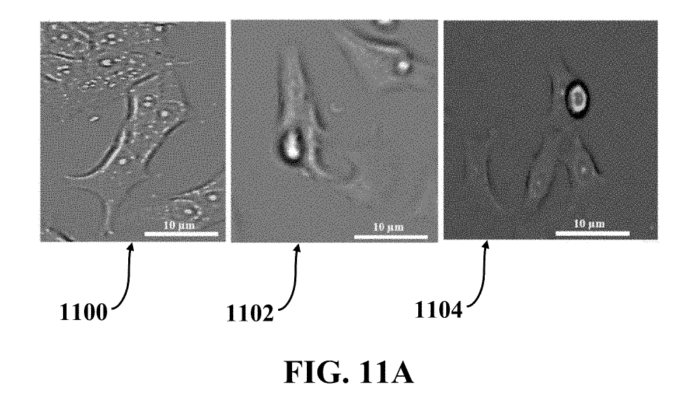

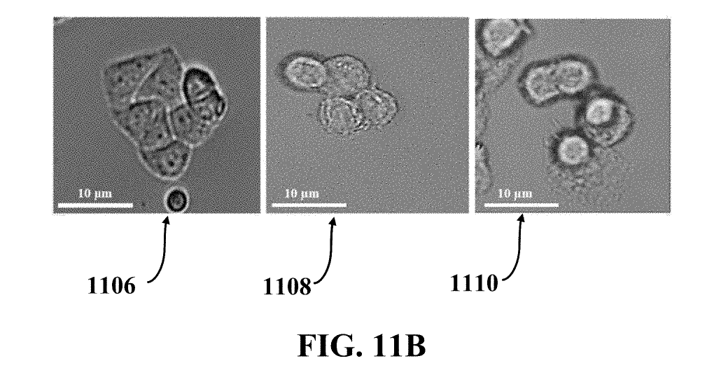

Optical microscopy images were taken from the MCF7 and MDA-MB468 cells in 16^{th} hour. FIGS. 11A and 11B show comparative optical images of MCF7 and MDA-MB468 cells cultured in three different pH values of 7.4 (left side image), 6.5 (middle image) and 5.5 (right side image), consistent with one or more exemplary embodiments of the present disclosure. It may be observed from these figures that lysosomes play the crucial role in lower pH values, due to activation of autophagy or because of self-eating.

Referring to FIG. 11A, optical microscopy images taken from the MCF7 in 16^{th} hour and in pH values of 7.4 (image 1100), 6.5 (image 1102) and 5.5 (image 1104) may show a trace of formed lysosomes in acidified MCF7 cells. In FIG. 11B, optical microscopy images taken from the MDA-MB468 cells in 16^{th} hour and in pH values of 7.4 (image 1106), 6.5 (image 1108) and 5.5 (image 1110) may be observed. The presence of lysosome in the cell as the indication of activated autophagy could be observed in MDA-MB468 cells. Moreover, the image 1110 taken from the MDA-MB468 cells in pH 5.5 may show the entrance of lysosome to form Autolysosome in the cells which activated self-eating mechanism in autophagy and induced apoptosis in the cell.

### Example 5: Western Blot and Zymography Analyses of Acidified Cells

In this example, the expression of LC3 associated proteins, as the major pH dependent protein which play the crucial role in autophagy in both MCF7 and MDA-MB468 cells, was investigated. After the specified treatment, cells were collected and the protein extraction was conducted mainly based on LC3 proteins. At first, the protein concentration was calculated by Bradford's method. Equivalent amounts of protein were boiled for about 5 minutes and detached by SDS-PAGE; then, were transferred onto a PVDF membrane. The membrane was then blocked with about 5% nonfat dry milk in Tris-Buffered-Saline with Tween (TBST) for about 1 hour at room temperature and incubated with suitable primary antibodies overnight at about 4° C. Subsequently, the membrane was washed with TBST and incubated with appropriate secondary antibody for about 1 hour at room temperature. After three washing steps with TBST, the proteins were observed employing the electrochemical luminescence (ECL) reagent. Analysis of the integrated density of the resultant protein bands was performed by Image J software.

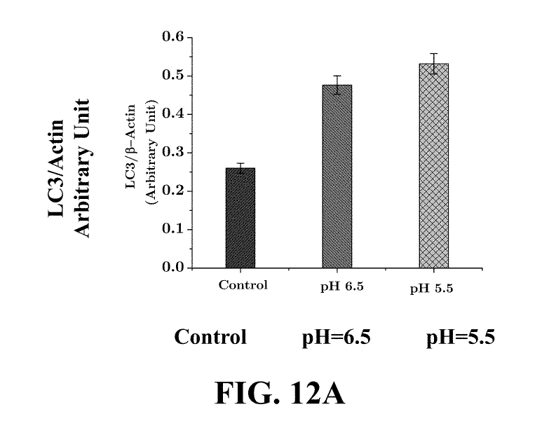

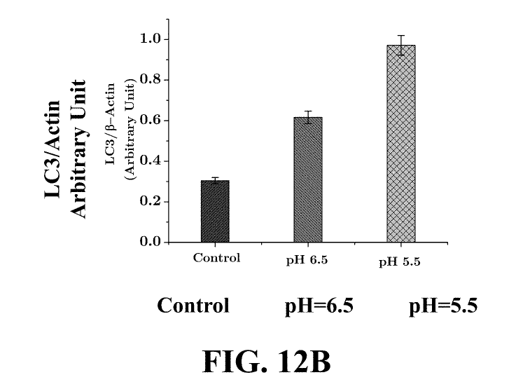

FIGS. 12A and 12B show comparative Western blotting profile of MCF7 (FIG. 12A) and MDA-MB468 (FIG. 12A) cells incubated in three different pH values of 7.4 (Control), 6.5, and 5.5 compared based on the expression LC3 associated proteins, consistent with one or more exemplary embodiments of the present disclosure. The expression LC3 associated proteins may be an important indicator of activation of autophagy. Beta actin was investigated as the reference value. Suitable expression of LC3 in MDA-MB468 cells post cultured in acidic revealed the appropriate activation of autophagy in such cells. The results of Western blot corroborated the expression of LC3 associated proteins. But the interesting point was that the level of LC3 in MCF7 cells maintained in pH=6.5 was more than the suitable range for autophagy of this type of cells. This indicated the acidosis of such cells. In contrast, this level in MDA-MB468 cells with similar maintaining parameters was beneath the suitable level relative to beta-actin in this cells for activation of autophagy. This would support the role of autophagy in maintaining the vitality of metastatic cells in moderate acidic ambient in less than about 12 hours. No trace of LC3 was observed in MCF10 cells (not shown here) that were tested in a similar way.

To ensure if the metastatic cells maintained their invasive behavior in acidic pH, MMP based Zymography was conducted on the MDA-MB468 cells that were maintained in different pH values of 7.4 (Control), 6.5, and 5.5 for about 4 hours. Gelatinase Zymography was performed in about 10% NOVEX Pre-Cast SDS Polyacrylamide Gel in the presence of about 0.1% gelatin under non-reducing conditions. Culture media (about 50 μl) were mixed with sample buffer and loaded for SDS-PAGE with tris glycine SDS buffer. Samples were not boiled before electrophoresis. Following electrophoresis, the gels were washed twice in about 2.5% TritonX-100 for about 30 minutes at room temperature to remove SDS. The gels were then incubated at about 37° C. overnight in substrate buffer containing about 50 mM Tris-HCl and about 10 mM CaCl₂ at pH of about 8.0 and stained with about 0.5% Coomassie Blue 8250 in about 50% methanol and about 10% glacial acetic acid for about 30 minutes and de-stained. Upon renaturation of the enzyme, the gelatinases digest the gelatin in the gel and give clear bands against an intensely stained background. Protein standards were run concurrently and approximate molecular weights were determined by plotting the relative motilities of known proteins.

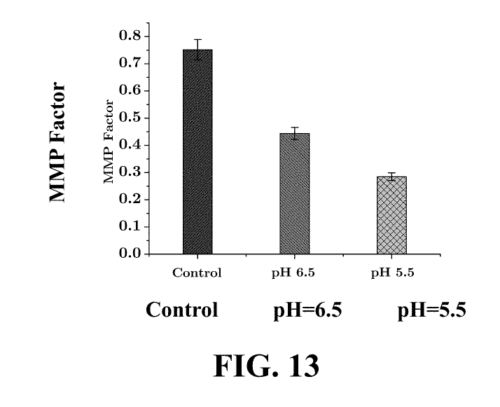

FIG. 13 shows Zymography results based on the expression of MMP2 for MDA-MB468 cells maintained in three different pH values of 7.4 (Control), 6.5, and 5.5 for about 4 hours, consistent with one or more exemplary embodiments of the present disclosure. Matrix metalloproteinase (MMPs) has an important role in metastasis. The results of Zymography indicated the reduced expression of MMP in acidic pH which revealed the decrease in their invasive ability during acidification.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1- A method for detecting a metastasis state of biological cells, the method comprising:
seeding a plurality of biological cells onto an array of electrodes of an electrical cell-substrate impedance sensor (ECIS) by dropping a cell suspension onto the array of electrodes, the cell suspension comprising the plurality of biological cells in a cell culture medium;
forming a plurality of cultured biological cells attached onto the array of electrodes by maintaining the ECIS in an incubator;
reducing pH value of an extracellular media around the plurality of cultured biological cells to a pH value between 6.2 and 6.7 by dropping an acidic solution onto the array of electrodes;
activating an intracellular phenomenon due to reducing pH value of the extracellular media around the plurality of cultured biological cells, the intracellular phenomenon comprising one of:
autophagy phenomenon in metastatic cells, and
cell's proliferation reduction and/or apoptosis in non-metastatic cells.
monitoring an electrical signal of the plurality of cultured biological cells for a pre-determined period of time by measuring a set of time-lapse electrical signals from the array of electrodes, comprising:
applying an electrical voltage to the array of electrodes; and
extracting the set of time-lapse electrical signals from the array of electrodes; and
determining metastasis state of the plurality of biological cells based on the monitored electrical signals, comprising:
identifying a metastatic state for the plurality of biological cells by detecting an increasing trend in the set of time-lapse electrical signals over time, wherein the increasing trend occurs responsive to activation of the autophagy phenomenon or identifying a non-metastatic state for the plurality of biological cells by detecting a decreasing trend in the set of time-lapse electrical signals over time, wherein the decreasing trend occurs responsive to activation of the cell's proliferation reduction and/or apoptosis phenomenon.

2- The method of claim 1, wherein identifying the non-metastatic state for the plurality of biological cells comprises identifying the plurality of biological cells comprising at least one of healthy cells, primary cancer cells, and combinations thereof.

3- The method of claim 1, wherein monitoring the electrical signal of the plurality of cultured biological cells for the pre-determined period of time comprises:
measuring the set of time-lapse electrical signals from the array of electrodes, comprising:
applying the electrical voltage to the array of electrodes; and
extracting the set of time-lapse electrical signals from the array of electrodes; and
recording the set of time-lapse electrical signals measured from the array of electrodes.

4- The method of claim 1, wherein the set of time-lapse electrical signals comprises a set of electrical impedances of the plurality of cultured biological cells.

5- The method of claim 1, wherein the pre-determined period of time comprises at least 8 hours after reducing pH value of the extracellular media around the plurality of cultured biological cells.

6- The method of claim 1, wherein the set of time-lapse electrical signals comprises a set of electrical impedance values measured every 2 hours after reducing pH value of the extracellular media around the plurality of cultured biological cells.

7- The method of claim 1, wherein applying the electrical voltage to the array of electrodes comprises applying a voltage ranging between 200 mV and 500 mV onto the array of electrodes.

8- The method of claim 7, wherein the electrical voltage is applied with a frequency ranging between 200 Hz and 100 kHz.

9- The method of claim 1, wherein monitoring the electrical signal of the plurality of cultured biological cells for a pre-determined period of time is done through a system, the system comprising:
a sensor package, comprising the ECIS;
an electrical readout board connected to the ECIS via coaxial wires, the electrical readout board configured to apply the electrical voltage to the array of electrodes, the electrical readout board further configured to extract the set of time-lapse electrical signals from the array of electrodes; and
a data processor connected to the electrical readout board via an electrical connector, the data processor configured to record the set of time-lapse electrical signals extracted by the electrical readout board.

10- The method of claim 1, wherein maintaining the ECIS in the incubator comprises maintaining the ECIS with the cell suspension dropped onto the array of electrodes in a CO₂ incubator for a time interval between 2 hours and 5 hours.

11- The method of claim 10, wherein the CO₂ incubator comprises 5% CO₂ and 95% clean air.

12- The method of claim 1, wherein the array of electrodes comprises an array of gold electrodes with a comb-shaped pattern, and
wherein each electrode of the array of electrodes comprises a plurality of silicon nanowires (SiNWs) covered onto each gold electrode.

13- The method of claim 12, wherein the array of electrodes comprises a plurality of electrodes with an equal width ranging between 10 μm and 100 μm.

14- The method of claim 13, wherein the array of electrodes comprises a first electrode and a second electrode located next to the first electrode, a distance between the first electrode and the second electrode ranging between 10 μm and 100 μm.

15- The method of claim 1, wherein non-metastatic cells comprise at least one of healthy cells, primary cancer cells, and combinations thereof.

16- A method for metastasis diagnosis, comprising:
seeding a plurality of biological cells suspicious to be metastatic onto an array of electrodes of an electrical cell-substrate impedance sensor (ECIS) by dropping a cell suspension onto the array of electrodes, the cell suspension comprising the plurality of biological cells in a cell culture medium;
forming a plurality of cultured biological cells attached onto the array of electrodes by maintaining the ECIS in an incubator;
reducing pH value of an extracellular media around the plurality of cultured biological cells to a pH value between 6.2 and 6.7 by dropping an acidic solution onto the array of electrodes;
activating autophagy phenomenon in metastatic cells due to reducing pH value of the extracellular media around the plurality of cultured biological cells;
monitoring an electrical signal of the plurality of cultured biological cells for a pre-determined period of time, comprising:
applying an electrical voltage to the array of electrodes; and
extracting a set of time-lapse electrical signals from the array of electrodes; and
diagnosing metastasis by detecting an increasing trend in the set of time-lapse electrical signals over time, wherein the increasing trend occurs responsive to activation of the autophagy phenomenon.

17- The method of claim 16, wherein diagnosing metastasis comprises detecting an increasing trend in the set of time-lapse electrical signals for a metastatic cell responsive to reducing pH value of the extracellular media around the plurality of cultured biological cells.

18- The method of claim 16, wherein diagnosing metastasis comprises detecting a reduction trend over time in the set of time-lapse electrical signals for a non-metastatic cell responsive to activation of a cell's proliferation reduction and/or apoptosis in non-metastatic cells due to reducing pH value of the extracellular media around the plurality of cultured biological cells.

19- The method of claim 16, wherein the non-metastatic cell comprises at least one of a normal cell, a primary cancer cell, and combinations thereof.
